(19) United States
(12) Patent Application Publication
(10) Pub. No.: US 2013/0165711 A1
(43) Pub. Date: Jun. 27, 2013

(54) PROCESS FOR SELECTIVE HYDROGENATION OF OLEFINIC FEEDSTOCKS WITH SWITCHABLE REACTORS INCLUDING AT LEAST ONE STAGE FOR SHORT-CIRCUITING A REACTOR

(71) Applicant: IFP ENERGIES NOUVELLES, RUEIL-MALMAISON CEDEX (FR)

(72) Inventors: Aurelie DANDEU, Saint-Just Chaleyssin (FR); Frederic BAZER-BACHI, Irigny (FR); Jean Louis AMBROSINO, Ternay (FR); Christophe BOYER, Charly (FR)

(73) Assignee: IFP ENERGIES NOUVELLES, RUEIL-MALMAISON CEDEX (FR)

(21) Appl. No.: 13/724,238

(22) Filed: Dec. 21, 2012

(30) Foreign Application Priority Data
Dec. 22, 2011 (FR) 11/62.310

(51) Int. Cl. C07C 5/03

(52) U.S. Cl. CPC C07C 5/03
USPC 585/251; 585/265

(57) ABSTRACT

This invention has as its object a process for selective hydrogenation of an unsaturated olefinic feedstock that comprises 3 or 4 carbon atoms, using at least two switchable fixed-bed reactors, each containing at least one catalytic bed and in which said feedstock successively passes through all of the reactors, and in which, each time that one of the reactors is deactivated, the point of introduction of the feedstock is moved downstream.

This invention has as its object a process for selective hydrogenation of an unsaturated olefinic feedstock that comprises 3 or 4 carbon atoms, using at least two switchable fixed-bed reactors, each containing at least one catalytic bed and in which said olefinic feedstock successively passes through all of the reactors and in which, each time that one of the reactors is deactivated, the point of introduction of the feedstock is moved downstream.

The primary thermal processes that generate unsaturated molecules are visbreaking and coking of petroleum residues, the steam-cracking of liquefied natural gases, gas condensates or naphthas, and the pyrolysis of carbons.

The most widely used catalytic processes that generate unsaturated compounds include the dehydrogenation of light paraffins, the catalytic reforming of gasolines, and the catalytic cracking of vacuum distillates.

Actually, the processes for conversion of hydrocarbons such as steam-cracking are performed at a high temperature and produce a large variety of unsaturated molecules such as ethylene, propylene, linear butenes, isobutene, pentenes, as well as unsaturated molecules that contain up to approximately 15 carbon atoms. In parallel, polyunsaturated compounds, such as acetylene, propadiene, and methyl acetylene (or propyne), 1-2 butadiene, and 1-3 butadiene, vinyl acetylene and ethyl acetylene, and other polyunsaturated compounds, are also formed.

The hydrogenation (partial or total, selective or not) of unsaturated hydrocarbons is an essential reaction of the operations for refining petroleum and for production of important intermediate products for petrochemistry. Actually, it is necessary to eliminate the most unsaturated hydrocarbons (alkynes, diolefins) of the light olefinic petroleum fractions to allow their use in petrochemistry or in the industry of polymers, where very high olefin purities are required. In the same way, these polyunsaturated compounds, gum precursors, should also be eliminated from gasoline fractions to ensure their stability.

Thus, the selective hydrogenation of unsaturated hydrocarbons makes it possible to selectively hydrogenate the polyunsaturated compounds that are present in the feedstock to be treated in such a way that the diolefinic compounds are partially hydrogenated in mono-olefins and that the styrene and indene compounds that are present in the gasoline fractions are partially hydrogenated into corresponding aromatic compounds.

The standard units for selective hydrogenation of unsaturated hydrocarbons generally comprise a primary hydrogenation section that comprises a catalytic fixed-bed reactor in which the liquid hydrocarbon feedstocks are brought into contact with the gaseous hydrogen (two-phase or one-phase reactor when all of the hydrogen can be dissolved in the feedstock). An intermediate heat exchanger can be placed downstream from the reactor to monitor the exothermy generated by the hydrogenation reaction. The hydrogenated olefins (comprising, for example, 200 to 5,000 ppm of methyl acetylene (MA) and propadiene (PD) for the fraction C3) are next separated from residual gaseous compounds by means of a separator tank and then optionally redirected toward a finishing section that comprises a finishing reactor or a “splitter” so as to improve the hydrogenation yield as well (until reaching MAPD contents that are less than 10 ppm).

The document FR-A-2810991 describes, for example, a process for hydrogenation of a C4 hydrocarbon fraction in a fixed-bed catalytic reactor, in which a portion of the hydrogenated product is recycled upstream from the hydrogenation reactor.

The hydrogenation reaction, however, brings about the formation of oligomers that can lead to the deposition of gums on the surface of the catalyst, bringing about its gradual deactivation. The result is that the catalyst is to be regenerated to recover its effectiveness. This is why this type of installation generally comprises a second fixed-bed reactor toward which the hydrogenation reaction is redirected when the first reactor is to be reactivated or regenerated.

The document U.S. Pat. No. 3,764,633 describes, for example, a process for isomerization of mono-olefins that contain a double bond placed at the molecule end that contains at least 4 carbon atoms and simultaneous selective hydrogenation of polyunsaturated compounds such as diolefins or acetylene compounds that are contained in the treated hydrocarbon fraction. The patent teaches that the process can use two reactors in parallel, each comprising, for example, two catalyst beds separated by a partition. When a reactor operates, the other is in a regeneration phase of the catalyst.

This second free reactor (also called “spare” in English terminology), although present in the installation, therefore is not used for the hydrogenation of olefins, even though the catalyst of the first reactor is not deactivated.

This invention therefore proposes to optimize the installations for selective hydrogenation of unsaturated hydrocarbons by proposing to use the reactors that are available in series in the primary hydrogenation section, without thereby having to interrupt the process for the regeneration of catalysts.

Thus, this application has as its object a process for selective hydrogenation of an unsaturated olefinic feedstock that comprises 3 or 4 carbon atoms, in which said unsaturated olefinic feedstock and a gaseous phase comprising hydrogen on a hydrogenation catalyst are passed, under hydrogenation conditions, into at least two fixed-bed hydrogenation reactors, each containing at least one catalytic bed, with said hydrogenation reactors being arranged in series to be used in a cyclical fashion by repeating, after a stage a) during which the feedstock successively passes through all of the hydrogenation reactions during a period that is at most equal to the deactivation time of one of said reactors,

successively stages b), b′) and c) defined below:
A stage b), during which the feedstock is introduced into the non-deactivated reactor that is located immediately downstream, relative to the direction of circulation of the feedstock, from the deactivated reactor, by short-circuiting the deactivated reactor, for a period that is at least equal to the deactivation time of said downstream reactor,
A stage b′), simultaneous to stage b), during which the catalyst from the deactivated reactor is regenerated and/or replaced by fresh catalyst,
A stage c), during which the feedstock passes through all of the hydrogenation reactors, the reactor whose catalyst was regenerated in stage b′) being reconnected in such a way as to be located downstream from the other reactors relative to the direction of circulation of the feedstock, and said stage being carried out for a period that is at most equal to the deactivation time of a reactor.

The process for selective hydrogenation according to the invention is implemented in a primary hydrogenation section in such a way as to produce hydrogenated olefins that comprise 200 to 5,000 ppm of MAPD, preferably 500 to 2,000 ppm, and more preferably on the order of 1,000 ppm. This process can optionally comprise an additional finishing stage by which the olefins will again be hydrogenated to finally not contain more than 1 ppm to 100 ppm of MAPD, preferably 1 to 10 ppm of MAPD.

The invention that is described here has as its object to improve the current processes for selective hydrogenation in a liquid phase of unsaturated olefinic feedstock comprising 3 or 4 carbon atoms. The use in series of all of the reactors that are present on the primary hydrogenation unit thus makes it possible to increase significantly the capacity of the unit without additional industrial investment. This process is therefore particularly useful when it is desired to increase the capacity of existing units (“revamping,” in English terminology) in an economical manner.

The process according to the invention also makes it possible to improve the selectivity of the hydrogenation reaction and to increase the duration of the cycle times by delaying the formation of oligomers (or gums), proportionately postponing the deactivation of the catalyst.

By way of example, over a period of two years, the process according to the invention makes it possible to make all of the reactors that are present in the unit operate for at least 23 months, by using the entire mass of the catalyst that is present in said reactors. In comparison, the standard installations operate with a single reactor and therefore have a reaction capacity that is half the size of the one available with the process according to the invention. This reactor that is used continuously is also to be regenerated and/or reactivated approximately every 2 years.

Unsaturated Olefinic Feedstock

The process according to the invention consists of the selective hydrogenation of unsaturated olefinic feedstocks that comprise 3 or 4 carbon atoms and that comprise in particular compounds comprising acetylene, diene, and alkenylaromatic functions.

The feedstock that is used within the framework of the process according to this invention is selected in particular from the group that consists of the C3 or C4 steam-cracking fractions.

These fractions that are obtained from steam-cracking, treated in the selective hydrogenation process according to the invention, comprise polyunsaturated hydrocarbons that contain at least 3 or 4 carbon atoms and that have a final boiling point that ranges up to 250° C. However, the specifications relative to the concentrations of these polyunsaturated compounds for the units of petrochemistry and polymerization are very low: on the order of 0.1 to 1,000 ppm by weight according to the applications in question.

The C3 steam-cracking fraction can comprise:
From 60 to 95% by weight of propylene, preferably 88% to 94% by weight,
From 1 to 8% by weight of propadiene (PD) and methyl acetylene (MA), preferably 2 to 8% by weight,
the make-up to 100% being essentially propane.

In certain C3 fractions, between 0.1 and 2% by weight of C2 and C4 can also be present.

The specifications are on the order of 10 ppm by weight of MAPD (methyl acetylene and propadiene) for “chemical”-quality propylene and less than 10 ppm by weight, and even up to 1 ppm by weight, for the “polymerization” quality.

The C4 steam-cracking fraction contains between 30 and 50% by weight of butadiene. In some C4 fractions, between 0.1 and 2% by weight of C3 and C5 can also be present.

The invention pertains particularly well to the selective hydrogenation of an unsaturated olefinic feedstock and comprises olefins that have 3 carbon atoms, with the olefinic feedstock preferably comprising 60% to 95% by weight of propylene, and 2 to 8% by weight of polyunsaturated compounds such as methyl acetylene (MA) and propadiene (PD), with the make-up to 100% being essentially propane.

This hydrogenation can be done in a gas-liquid flow under bubbling conditions with a volumetric vaporization rate at the reactor inlet that ranges from 1 to 50% by volume, preferably 5 to 30% by volume, or in a liquid-only flow, with a volumetric vaporization rate at the reactor inlet that ranges from 0 and 5% by volume.

Hydrogenation Conditions

Within the framework of the process according to the invention, the unsaturated olefinic feedstock is brought into contact with a gaseous phase that comprises hydrogen in the presence of a hydrogenation catalyst, under conditions in particular of temperature, pressure, and hourly volumetric flow rate (VVH), making hydrogenation possible.

In particular, the selective hydrogenation process is advantageously performed under pressure, in a liquid phase, and in the presence of hydrogen.

The selective hydrogenation process according to the invention is preferably implemented in each reactor at a temperature that ranges from 10° C. to 80° C., more preferably from 15 to 65° C.

The pressure in each reactor is preferably from 10 to 40 bar, more preferably from 15 to 35 bar, keeping in mind that 1 bar=0.1 MPa (0.1 mega pascal) in the international system.

The overall hourly volumetric flow rate (VVH), defined as the ratio of the volumetric flow rate of the fresh feedstock at 15° C. to the total volume of catalyst present in all of the switchable reactors used, is generally from 2 h⁻¹ to 100 h⁻¹, preferably from 5 h⁻¹ to 50 h⁻¹, and more preferably from 10 h⁻¹ to 30 h⁻¹.

Gaseous Phase

Within the framework of the process according to the invention, the unsaturated olefinic feedstock is brought into contact with a gaseous phase that comprises hydrogen.

The gaseous feedstock is often composed of a mixture of hydrogen and at least one other gas, inert for the reaction according to the purification process that is used. This other gas can, for example, be selected from the group that is formed by methane, ethane, propane, butane, nitrogen, argon, carbon monoxide (several ppm), and carbon dioxide. This other gas is preferably methane or propane and is more preferably free of carbon monoxide.

In particular, when the selective hydrogenation process according to the invention does not comprise an additional finishing stage, the amount of hydrogen is preferably slightly in excess relative to the stoichiometric value (relative to the desired MAPD conversion), making possible the selective hydrogenation of polyunsaturated compounds that are present in the hydrocarbon feedstock. In this embodiment, the hydrogen excess is generally between 1 and 50% by weight, preferably between 1 and 30% by weight.

The proportion of hydrogen in the gaseous feedstock is in particular from 60% to 100% by weight, and most often from 80% to 99.99% by weight, with the make-up to 100% being one of the inert gases cited above.

According to a particularly preferred method of the invention, the gaseous phase consists of hydrogen at 100% by weight.

Hydrogenation Catalyst

The catalyst that is used in the hydrogenation reactors according to the invention can preferably comprise at least one metal of group VIII, more preferably palladium.

The metal of group VIII, preferably palladium, can preferably be deposited in a crust on the periphery of the substrate (balls, extrudates), in such a way that the MAPD compounds react on the surface of said catalyst and are converted into propylene. The distribution on the crust is well known to one skilled in the art and makes possible a better selectivity of the catalyst in that the MAPD are converted into propylene, but the propylene is not hydrogenated into propane.

In a particularly preferred manner, the catalyst that is used in the hydrogenation reactors according to the invention comprises from 0.01 and 2% by weight of palladium, preferably from 0.03 and 0.8% by weight.

Preferably, the selective hydrogenation catalysts also comprise at least one metal that is selected from the group that consists of alkalines and alkaline-earth compounds.

The alkaline metal is generally selected from the group that consists of lithium, sodium, potassium, rubidium and cesium, preferably lithium, sodium and potassium, in a very preferred manner sodium and potassium. In an even more preferred manner, the alkaline metal is sodium.

The alkaline-earth metal is generally selected from the group that consists of magnesium, calcium, strontium and barium, preferably magnesium and calcium, and in a very preferred manner magnesium.

The sum of the contents of alkaline metals or alkaline-earth compounds preferably ranges from 0.05 to 5% by weight, and preferably from 0.1 to 2% by weight, relative to the total weight of the catalyst.

The catalyst according to the invention in particular comprises a porous substrate that comprises at least one refractory oxide that is preferably selected from among the oxides of metals of groups IIA, IIIB, IVB, IIIA and IVA according to the CAS notation of the periodic table. In a preferred manner, said substrate is formed by at least one simple oxide that is selected from among alumina (Al₂O₃), silica (SiO₂), titanium oxide (TiO₂), cerium oxide (CeO₂), and zirconia (ZrO₂).

In a preferred manner, said substrate is selected from among aluminas, silicas, and silica-aluminas.

In a particularly preferred manner, the porous substrate is an alumina.

According to a particular embodiment, the specific surface area of the substrate ranges from 20 to 210 m²/g, preferably from 20 to 160 m²/g, and more preferably from 20 to 150 m²/g.

The porous substrate can come in particular in the shape of balls, trilobes, extrudates, pellets, or irregular and non-spherical agglomerates whose specific shape can result from a crushing stage.

In a very advantageous manner, the substrate comes in the form of balls or extrudates. In an even more advantageous manner, said substrate comes in the form of balls.

The pore volume of the substrate is generally between 0.1 and 1.5 cm³/g, preferably between 0.5 and 1.3 cm³/g.

Preferably, the catalyst that is used can comprise, in addition, at least one doping agent, belonging to column IB of the periodic table, which can preferably be selected from the group that is formed by gold, silver, and copper, and more preferably silver.

The doping agent can be present in a quantity that generally ranges from 1 to 10,000 ppm by weight, preferably from 1 to 5,000 ppm by weight, and more preferably from 1 to 2,000 ppm by weight.

Hydrogenation Reactors

The process for hydrogenation according to the invention implements, in a primary hydrogenation section, at least two fixed-bed hydrogenation reactors, each containing at least one catalytic bed, whereby said hydrogenation reactors are arranged in series to be used in a cyclic manner.

In a more specific manner, the flow rate of the gaseous phase that comprises hydrogen adjusted at the inlet of each reactor in such a way as to obtain the desired overall MAPD conversion level [sic].

Actually, during the hydrogenation of the unsaturated olefinic fraction, the MAPD compounds react with hydrogen to form propylene. When hydrogen is in a molar excess relative to the MAPD (H₂/MAPD ratio>1), 100% of the desired overall MAPD conversion can be obtained.

In practice, a portion of the added hydrogen can be consumed in secondary reactions that lead to the formation of propane and oligomer.

When there is too little hydrogen, (H₂/MAPD ratio<1), only a portion of the desired overall MAPD conversion is effectively obtained.

Within the framework of the invention implementing several hydrogenation reactors in series, the desired MAPD conversion for each reactor is defined based on the desired overall MAPD conversion in the primary hydrogenation section (comprising the switchable reactors).

For example, to obtain a desired overall MAPD conversion of 80% in a primary hydrogenation section that comprises two reactors, the MAPD conversion can be 40% in each reactor.

In a particularly preferred manner, the catalyst mass is the same in each switchable reactor.

Thus, if X is the desired overall MAPD conversion in the primary hydrogenation section comprising the switchable reactors, the conversion x, for the n reactors is to be between 20 and 150% of X/n per reactor.

In the case of the example of a desired overall MAPD conversion of 80%, the conversion xᵢ for each reactor should be between 20% [(X=80%)/(n=2)] to 150% [(X=80%)/(n=2)], i.e., from 8% to 60% for each reactor.

Thus, the hydrogenation process according to the invention, implementing several reactors in the primary hydrogenation section, has as its objective an MAPD specification that ranges from 200 to 5,000 ppm, preferably from 500 to 2,000 ppm, and more preferably on the order of 1,000 ppm.

The gaseous phase that comprises hydrogen is preferably at least introduced at the top of the first reactor through which the feedstock passes and can advantageously also be introduced at the top of each hydrogenation reactor that is present in the primary hydrogenation unit.

By thus staging the introduction of the gaseous feedstock that comprises hydrogen between the different reactors, it is possible to introduce reduced quantities of hydrogen at the top of each reactor, which limits the risk of secondary reactions in each reactor. In addition, the introduction of a gaseous phase that comprises hydrogen at the top of each reactor and at a temperature that is close to ambient temperature (approximately 20° C.) makes it possible to lower the temperature of the feedstock that is introduced into the downstream reactor (the hydrogenation reaction being exothermic), thus limiting the vaporization of the olefinic feedstocks that is favorable to a better selectivity. The conjunction of the introduction of a small amount of hydrogen and an olefinic feedstock kept liquid brings about better solubilization of the hydrogen in the feedstock, in such a way that the reactor comes close to liquid one-phase conditions. These speeds that are close to the one-phase speed make it possible also to improve the selectivity of the hydrogenation reactions.

According to another embodiment, at least one of the hydrogenation reactors contains at least two catalyst beds. In this embodiment, the gaseous phase that comprises hydrogen can then be introduced partially mixed with the unsaturated olefinic feedstock before the first catalyst bed and partially before the following bed(s) contained in said reactor.

Finishing Section

Within the framework of this invention, in the case where it is desired to reduce the conversion level below 1,000 ppm (a preferred mode but not an exclusive one), a finishing section that comprises a finishing reactor or a splitter can advantageously be added at the outlet of the primary hydrogenation section that comprises the switchable reactors. The MAPD conversion conditions in the finishing unit are separate from those of the primary unit comprising at least two reactors.

According to this particular embodiment, the entirety or the non-recycled portion of the liquid phase containing the recovered hydrogenated olefinic feedstock can be sent to a finishing section that comprises
A “splitter” or (séparateur, French terminology) or
A “finishing reactor” preceded by a static mixer M2 for mixing again the hydrogenated olefinic feedstock with a gaseous phase that comprises hydrogen.

This finishing stage can make it possible also to reduce the MAPD content in the hydrogenated olefinic feedstock to a value that ranges from 1 to 100 ppm, preferably from 1 to 10 ppm.

Heat Exchanger (Cooler)

According to a preferred embodiment, the hydrogenation process according to the invention in particular can implement one or more heat exchanger(s) (cooler) between each reactor in such a way as to cool the effluent of the reactor that is located immediately upstream.

Thus, the gas/liquid mixture that is obtained from an upstream hydrogenation reactor passes through a heat exchanger E before being sent to the inlet of a reactor that is located downstream, relative to the direction of circulation of the feedstock, preferably from the reactor that is located immediately downstream. The temperature of the effluent that is obtained from the first reactor is thus reduced in such a way as to liquefy the olefins that are vaporized in the reactor.

This intermediate heat exchanger therefore makes it possible to monitor the exothermy generated by the hydrogenation reaction. The propylene selectivity will thus be improved.

When the process according to the invention implements one or more heat exchanger(s) (cooler), the gaseous phase that comprises hydrogen can be introduced upstream and/or downstream from said exchanger.

Regeneration/Reactivation

Within the framework of the invention, the catalysts of the hydrogenation reactors, in particular the first reactor that is brought into contact with the feedstock, gradually build up metals, oligomers, sediments, and other diverse impurities. When the catalysts are virtually saturated with metals and diverse impurities, the zones should be disconnected to carry out the replacement and/or the regeneration of catalyst(s). Preferably, the catalysts are then regenerated and/or replaced. The time between the beginning of the hydrogenation and the moment when one of the reactors is deactivated is called deactivation time. Although the deactivation time varies based on the feedstock, operating conditions and the catalyst(s) used, it is expressed in a general manner by a drop in the catalytic performance level (an increase of the concentration of metals and/or other impurities in the effluent), or an increase in the flow rate of hydrogen that is necessary for keeping hydrogenation constant. The temperature is measured continuously during the entire cycle on each of the reactors.

When one of the reactors begins to become deactivated, the feedstock is oriented toward the reactor located immediately downstream, relative to the direction of circulation of the feedstock. The deactivated reactor is thus regenerated or reactivated.

The regeneration can be carried out in particular at a temperature that ranges from 200 to 450° C., with a gradual rise by temperature rates of climb and with successive additions of water vapor (steam stripping) and oxygen (combustion).

For its part, the reactivation can be carried out at a lower temperature, encompassed between 100 and 200° C. (for example on the order of 150° C.), by treatment with a mixture of N₂ and H₂.

Such a regeneration procedure can make it possible in particular to limit the sintering of metal particles and the degradation of the substrate.

Static Mixer

According to a particular embodiment of the invention, the liquid feedstock and the gaseous phase that are introduced at the top of one of the reactors (in particular at the top of the primary hydrogenation section) advantageously pass through a static mixer before being introduced into the reactor, in such a way in particular as to mix the hydrogen well in the liquid hydrocarbon phase so as to ensure bubbling conditions in the reactor.

Recycling

According to a preferred embodiment of the invention, a portion of the gas/liquid effluent that is obtained from the hydrogenation section (i.e., obtained from the primary hydrogenation section when the process according to the invention does not comprise an additional finishing stage or obtained from the finishing section when the process comprises a finishing stage) can be sent (i.e., recycled) in a mixture with the feedstock to be hydrotreated.

According to another preferred embodiment of the invention, in the primary hydrogenation section, a portion of the gas/liquid effluent that is obtained from a downstream hydrogenation reactor can be returned (i.e., recycled) at the inlet of said reactor and/or a reactor that is located upstream, preferably a reactor that is located immediately upstream.

The objective of this recycling is to dilute the feedstock at the inlet of the reactor to limit the formation of oligomers (or gums).

When the process according to the invention implements recycling, the gaseous phase that comprises hydrogen can be introduced before and/or after the recycling is introduced.

### BRIEF DESCRIPTION OF THE DRAWINGS

FIG. 1 represents standard hydrogenation processes.

FIGS. 2-4 represent various embodiments of the invention.

### DETAILED DESCRIPTION

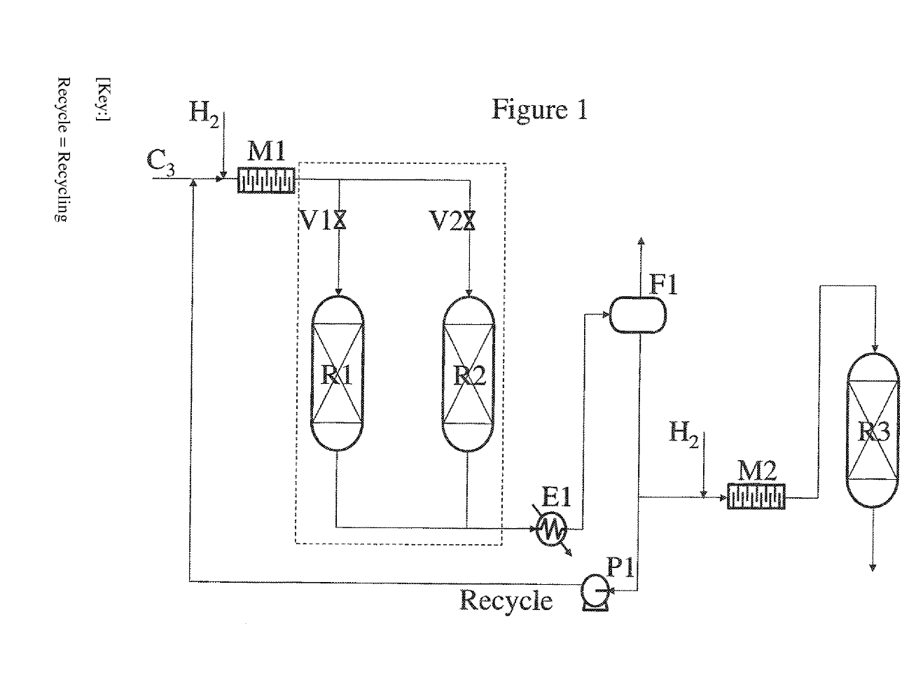

This invention is described in more detail using FIGS. 1 to 4.

This invention constitutes an improvement of the standard hydrogenation processes (primary hydrogenation section) as illustrated in FIG. 1.

The standard hydrogenation processes comprise, as FIG. 1 shows, two hydrogenation reactors:

A primary reactor R1 and a “spare” reactor R2 in parallel.

In this process, a C3 olefinic feedstock (C3 fraction) is introduced with the gaseous phase comprising hydrogen in a static mixer M1 in such a way as to thoroughly mix them. The mixture at the outlet of M1 is next sent to the top of the hydrogenation reactor R1, operated under hydrogenation conditions and comprising a hydrogenation catalyst. The valve V1 is thus open, and for its part, the valve V2 is closed.

The gas/liquid effluent that is obtained from R1 generally comprises less than 200 to 5,000 ppm of MAPD, preferably from 500 to 2,000 ppm, and more preferably on the order of 1,000 ppm.

This effluent next passes into a heat exchanger E1 for lowering its temperature in such a way as to liquefy the olefins that are vaporized in the reactor.

This mixture is next sent to a tank F1 starting from which a liquid phase is recovered that comprises the selectively hydrogenated olefinic feedstock.

A portion of the liquid phase that contains the hydrogenated olefinic feedstock can next be recycled at the inlet of the reactor R1, to be mixed with the feedstock to be hydrogenated, with the gaseous phase comprising hydrogen.

According to a particular embodiment, the liquid phase that contains the hydrogenated olefinic feedstock that is obtained from the separation section F1 can be sent to a finishing section that comprises a finishing reactor R3 after having passed through a static mixer M2 to be mixed again with a gaseous phase that comprises hydrogen. This finishing stage can make it possible to lower again the MAPD content in the hydrogenated olefinic feedstock to a value that ranges from 1 ppm to 100 ppm, preferably from 1 to 10 ppm.

When the reactor R1 begins to be deactivated, the feedstock that is obtained from the mixer M1 is oriented toward the second reactor R2. For this purpose, the valve V2 is open, and the valve V1 is closed. The feedstock is thus treated in the reactor R2 while R1 is cleaned and its catalyst regenerated.

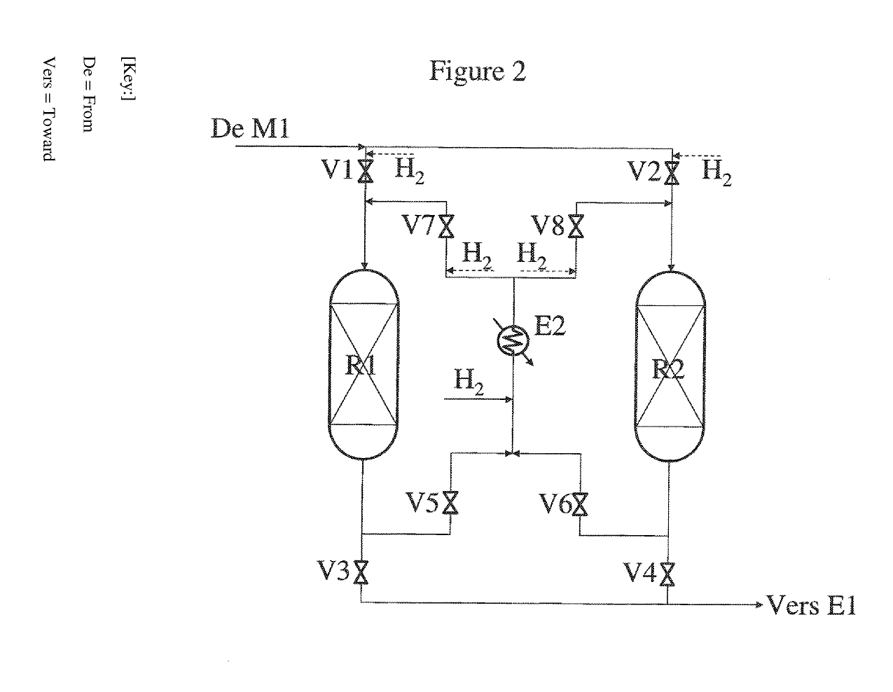

This invention, as illustrated in FIG. 2, proposes using continuously the two reactors R1 and R2 that are present in the unit of FIG. 1.

Within the framework of the selective hydrogenation process according to the invention, a C3 unsaturated olefinic feedstock (C3 fraction) and a gaseous phase comprising hydrogen are sent, under hydrogenation conditions, to a hydrogenation catalyst, in at least two fixed-bed hydrogenation reactors R1 and R2, each containing at least one catalytic bed, whereby said hydrogenation reactors R1 and R2 are arranged in series to be used in a cyclic manner by repeating, after a “stage a” during which the feedstock successively passes through the reactor R1 and then the reactor R2, for a period that is at most equal to the deactivation time of R1,

successively the following stages b), b′), and c):
A “stage b,” during which the feedstock passes through only the reactor R2, with the reactor R1 being short-circuited for regeneration and/or replacement of the catalyst,
A “stage b′,” simultaneous to stage b), during which the catalyst of the reactor R1 is regenerated and/or replaced by fresh catalyst,
A “stage c,” during which the feedstock successively passes through the reactor R2, and then the reactor R1.

During stage a) of the process, the feedstock, previously mixed with the gaseous phase comprising hydrogen, is introduced via the line that comprises a valve V1 that is open toward the reactor R1. During this period, the valves V2, V3, V6 and V7 are closed.

The effluent of the reactor R1 is sent via a pipe that comprises an open valve V5 and a pipe comprising an open valve V8 in the reactor R2.

The effluent of the reactor R2 is next evacuated via the pipe comprising an open valve V4.

During stage b) of the process, the valves V1, V3, V5, V6, V7 and V8 are closed, and the feedstock is introduced via the line that comprises a valve V2 that is open toward the reactor R2. During this period, the effluent of the reactor R2 is evacuated via the pipe that comprises an open valve V4.

During this stage b), a quantity of feedstock of 50 to 100% by weight of the quantity of fresh feedstock introduced in stage a), preferably from 60 to 100% by weight of the fresh feedstock, and more preferably from 80 to 100% by weight, is introduced into the non-deactivated reactor R2. The hydrogen flow rate will be adjusted to comply with the desired MAPD conversions.

During stage c), the valves V1, V4, V5 and V8 are closed, and the valves V2, V3, V6 and V7 are open. The feedstock is introduced via the line that comprises a valve V2 that is open toward the reactor R2. The effluent of the reactor R2 is sent via the pipe that comprises a valve V6 that is open in the reactor R1. The effluent of the reactor R1 is next evacuated via the pipe that comprises an open valve V3.

During stage d), the valves V2, V4, V5, V6, V7 and V8 are closed, and the valves V1 and V3 are open. The feedstock is introduced via the line that comprises a valve V1 that is open toward the reactor R1. During this period, the effluent of the reactor R1 is evacuated via the pipe that comprises an open valve V3.

The cycle then begins again. Table 1 reflects these changes in terms of the opening and closing of different valves.

TABLE 1
Operation of Valves Around Two Primary Reactors.

| Cycle Stage | | V1 | V2 | V3 | V4 | V5 | V6 | V7 | V8 |
|---|---|---|---|---|---|---|---|---|---|
| a | R1 + | Open | Closed | Closed | Open | Open | Closed | Closed | Open |
| | R2 | | | | | | | | |
| b | R2 | Closed | Open | Closed | Open | Closed | Closed | Closed | Closed |
| c | R2 + | Closed | Open | Open | Closed | Closed | Open | Open | Closed |
| | R1 | | | | | | | | |
| b | R1 | Open | Closed | Open | Closed | Closed | Closed | Closed | Closed |

The gas/liquid effluent that is obtained from this hydrogenation section generally comprises less than 200 to 5,000 ppm by weight of MAPD compounds, preferably from 500 to 2,000 ppm, and more preferably on the order of 1,000 ppm.

According to a preferred embodiment, during stages a) or c), the gas/liquid mixture that is obtained from an upstream hydrogenation reactor passes through a heat exchanger E2 (cooler) before being sent to the inlet of a reactor that is located downstream, relative to the direction of circulation of the feedstock, preferably from the reactor that is located immediately downstream. The temperature of the effluent that is obtained from the first reactor is thus lowered in such a way as to liquefy the olefins that are vaporized in the reactor. This intermediate heat exchanger therefore makes it possible to monitor the exothermy that is generated by the hydrogenation reaction.

According to an embodiment, the gaseous phase that comprises hydrogen is introduced at the top of the reactor R1. According to a preferred embodiment, the gaseous phase that comprises hydrogen is introduced in part at the top of the reactor R1 and in part at the top of the reactor R2, before or after the exchanger (cooler E2).

According to a particular embodiment, it is possible to provide an additional intake of gaseous phase comprising hydrogen at the inlet of each of the reactors (at the line comprising the valve V2) in the event of damage of the first intake of gaseous phase comprising hydrogen located at the inlet of the first reactor (at the line comprising the valve V1).

According to another preferred embodiment, it is possible to add to the stages of the above-described process an additional stage a′), before stage b), during which a portion of the unsaturated olefinic feedstock supplies an upstream reactor and the remainder of the feedstock supplies at least one of the reactors located downstream from said upstream reactor, for a period that is at most equal to the deactivation time of one of said reactors, which makes possible in particular a gradual switching of the reactors.

According to still another preferred embodiment, a portion of the gas/liquid effluent that is obtained from a downstream hydrogenation reactor can be returned (i.e., recycled) at the inlet of said reactor and/or a reactor that is located upstream, preferably a reactor that is located immediately upstream.

In this embodiment, at the outlet of each reactor, a portion (or a percentage) of the gas/liquid effluent can be recycled.

Thus, in FIG. 2, when the feedstock successively passes through the reactor R1 and the reactor R2 (“stage a”), a percentage of the gas/liquid effluent at the outlet of the reactor R1 can be recycled at the inlet of said reactor R1 via the opening of the valve V7.

In the same way, when the feedstock successively passes through the reactor R2 and then the reactor R1 (“stage c”), a percentage of the gas/liquid effluent at the outlet of the reactor R2 can be recycled at the inlet of said reactor R2 via the opening of the valve V8.

The recycling rate, defined as the ratio of the mass flow rate of recycling to the mass flow rate of feedstock entering the reactor can be in particular from approximately 1:10 to approximately 3:1, preferably 1:3 to 2:1.

Just as in the hydrogenation processes of the prior art that are illustrated in FIG. 1, the gas/liquid mixture obtained from the hydrogenation unit according to the invention can next be sent into a flask from which a gaseous phase, an aqueous phase that is eliminated, and a liquid phase comprising the hydrogenated olefinic feedstock are recovered.

A portion of the liquid phase containing the hydrogenated olefinic feedstock can next be sent to the inlet of the reactor R1 to be recycled in a mixture with the feedstock that is to be hydrogenated.

The recycling rate, defined as the ratio of the volumetric flow rate of recycling to the volumetric flow rate of the feedstock entering the first reactor, can then be from approximately 1:10 to approximately 3:1, preferably 1:3 to 2:1.

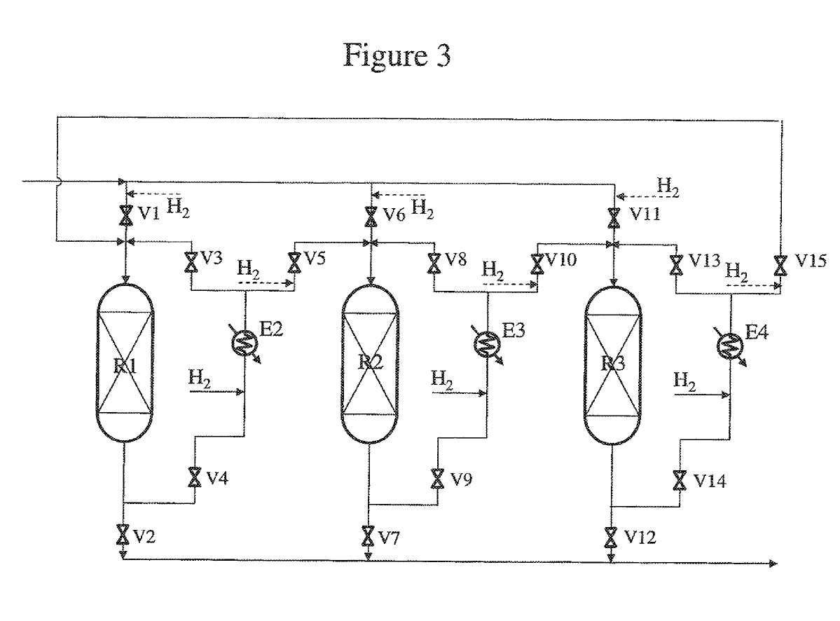

FIG. 3 illustrates a particular embodiment of the invention, in which it is proposed to use three reactors R1, R2 and R3 continuously.

After a “stage a,” during which the feedstock successively passes through the reactor R1, the reactor R2, and then the reactor R3, this hydrogenation process comprises a series of successive cycles:

1^{st} Cycle:
A “stage b,” during which the feedstock passes through the reactor R2 and then the reactor R3, with the reactor R1 being short-circuited for regeneration and/or replacement of the catalyst,
A “stage b′,” simultaneous to stage b, during which the reactor R1 is regenerated and/or its catalyst is replaced,
A “stage c,” during which the feedstock successively passes through the reactor R2, the reactor R3, and then the reactor R1,

2^{nd} Cycle:
A new “stage b,” during which the feedstock passes through the reactor R3 and then the reactor R1, with the reactor R2 being short-circuited for regeneration and/or replacement of the catalyst,
A new “stage b′,” simultaneous to stage b, during which the reactor R2 is regenerated and/or its catalyst is replaced,
A new “stage c,” during which the feedstock successively passes through the reactor R3, the reactor R1, and then the reactor R2,

3^{rd} Cycle:
A new “stage b,” during which the feedstock passes through the reactor R1 and then the reactor R2, with the reactor R3 being short-circuited for regeneration and/or replacement of the catalyst,
A new “stage b′,” simultaneous to stage b, during which the reactor R3 is regenerated and/or its catalyst is replaced,
A new “stage c,” during which the feedstock successively passes through the reactor R1, the reactor R2, and then the reactor R3 (corresponding to stage a)).

The cycle then begins again. Table 2 reflects these changes in terms of the opening and closing of different valves.

TABLE 2
Operation of Valves Around the Three Primary Reactors.

| Cycle Stage | | V1 | V2 | V3 | V4 | V5 | V6 | V7 | V8 | V9 | V10 | V11 | V12 | V13 | V14 | V15 |
|---|---|---|---|---|---|---|---|---|---|---|---|---|---|---|---|---|
| a | R1 + | O* | C* | C | O | O | C | C | C | O | O | C | O | C | C | C |
| | R2 + | | | | | | | | | | | | | | | |
| | R3 | | | | | | | | | | | | | | | |
| b | R2 + | C | C | C | C | C | O | C | C | O | O | C | O | C | C | C |
| | R3 | | | | | | | | | | | | | | | |
| c | R2 + | C | O | C | C | C | O | C | C | O | O | C | C | C | O | O |
| | R3 + | | | | | | | | | | | | | | | |
| | R1 | | | | | | | | | | | | | | | |
| b | R3 + | C | O | C | C | C | C | C | C | C | C | O | C | C | O | O |
| | R1 | | | | | | | | | | | | | | | |
| c | R3 + | C | C | C | O | O | C | O | C | C | C | O | C | C | O | O |
| | R1 + | | | | | | | | | | | | | | | |
| | R2 | | | | | | | | | | | | | | | |
| b | R1 + | O | C | C | O | O | C | O | C | C | C | C | C | C | C | C |
| | R2 | | | | | | | | | | | | | | | |

*O = Open
*C = Closed

According to still another preferred embodiment, a portion of the gas/liquid effluent that is obtained from a downstream hydrogenation reactor can be returned (i.e., recycled) at the inlet of said reactor.

It is thus possible to dilute the feedstock at the inlet of the upstream reactor to prevent the formation of oligomers (or gums).

In this embodiment, at the outlet of each reactor, a portion (or a percentage) of the gas/liquid effluent can be recycled.

Thus, in FIG. 3, when the feedstock successively passes through the reactor R1, the reactor R2, and then the reactor R3 (“stage a”), a percentage of the gas/liquid effluent at the outlet of the reactor R1 can be recycled at the inlet of said reactor R1 via the opening of the valve V3 and/or a percentage of the gas/liquid effluent at the outlet of the reactor R2 can be recycled at the inlet of said reactor R2 via the opening of the valve V8 and/or a percentage of the gas/liquid effluent at the outlet of the reactor R3 can be recycled at the inlet of said reactor R3 via the opening of the valve V13.

The recycling rate, defined as the ratio of the volumetric flow rate of recycling to the volumetric flow rate of the feedstock entering the reactor can in particular be from approximately 1:10 to approximately 3:1, and preferably 1:3 to 2:1.

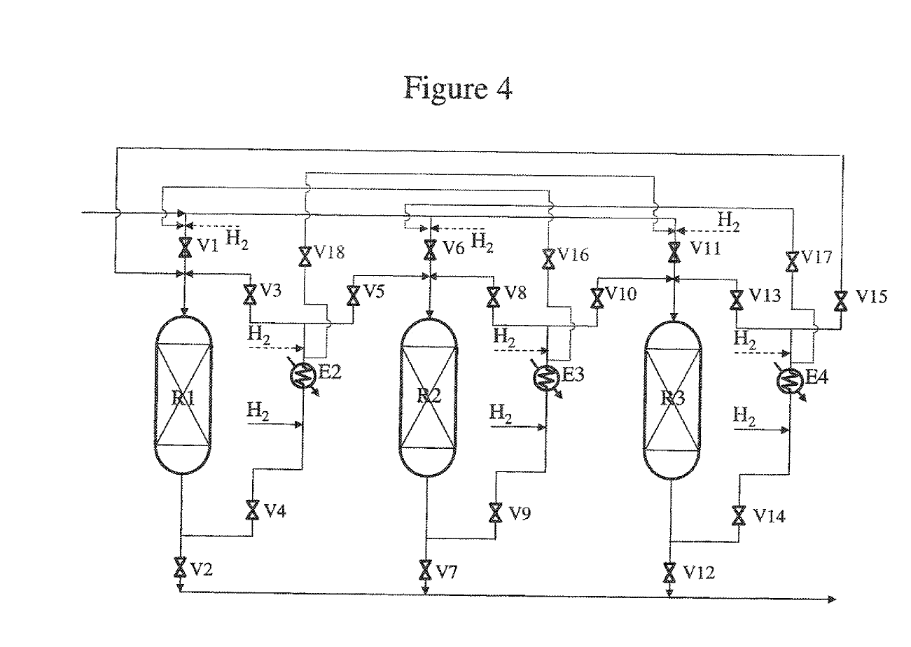

FIG. 4 illustrates a particular embodiment of the invention that is illustrated in FIG. 3, in which it is proposed to use three reactors R1, R2 and R3 continuously and where an internal recycling is provided for each reactor.

Actually, according to still another preferred embodiment of the invention, a portion of the gas/liquid effluent obtained from a downstream hydrogenation reactor can be returned (i.e., recycled) at the inlet of said reactor (as illustrated in FIG. 3) and/or a reactor that is located upstream, preferably at the inlet of the first reactor through which the feedstock passes in the cycle being considered.

The recycling can be carried out according to a first option, after the exchanger but before the injection of hydrogen (then illustrated in dotted lines in FIG. 4).

Thus, in FIG. 4, when the feedstock successively passes through the reactor R1, the reactor R2, and then the reactor R3 (“stage a”), a percentage of the gas/liquid effluent at the outlet of the reactor R2 can be recycled at the inlet of said reactor R1 via the opening of the valve V16.

In the same manner, when the feedstock successively passes through the reactor R2, the reactor R3 and then the reactor R1 (“stage c”), a percentage of the gas/liquid effluent at the outlet of the reactor R3 can be recycled at the inlet of said reactor R2 via the opening of the valve V17.

Finally, when the feedstock successively passes through the reactor R3, the reactor R1, and then the reactor R2 (“stage c”), a percentage of the gas/liquid effluent at the outlet of the reactor R1 can be recycled at the inlet of said reactor R3 via the opening of the valve V18.

The recycling rate, defined as the ratio of the volumetric flow rate of recycling to the volumetric flow rate of feedstock entering the reactor, can be in particular from approximately 1:10 to approximately 3:1, preferably 1:3 to 2:1.

FIGS. 2, 3 and 4 illustrate embodiments of the process according to the invention in which 2 or 3 hydrogenation reactors are used in series in the primary hydrogenation section. These embodiments are non-limiting, and the process according to the invention could thus be implemented with a non-limited number of n reactors in series.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding French application No. 11/62.310, filed Dec. 22, 2011 are incorporated by reference herein.

### EXAMPLES

### Example 1

### According to the Prior Art

An unsaturated olefinic feedstock that comprises 91% propylene, 4% propane, 3% methyl acetylene and 2% propadiene was treated by a hydrogenation process as illustrated in FIG. 1, under the following operating conditions:
Flow rate of feedstock: 50 m³/h
Composition of the gaseous phase comprising hydrogen: 93% H2, 7% CH4
Total flow rate of hydrogen: 751 Nm³/h (H2+CH4)
VVH, defined as the ratio of the volumetric flow rate of fresh feedstock at 15° C. to the volume of catalyst: 15 h⁻¹.
Volume of catalyst of 3 m³ in a reactor with a 1,000 mm diameter (primary reactor, active catalyst),
Reserve of catalyst of 3 m³ in a reactor with a 1,000 mm diameter (spare reactor, inactive catalyst),
Flow rate of recycling: 49.3 m³/h
Absolute pressure at the reactor inlet: 23 bar.
Temperature at the reactor inlet: 30° C.

The objective in this example is to lower the content of MAPD (methyl acetylene and propadiene) to 1,000 ppm at the outlet of the reactor.

In this embodiment, a single reactor is used for hydrogenation. 100% of the desired MAPD conversion (intake concentration brought to 1,000 ppm) should therefore be done in this reactor.

The composition of the feedstock at the outlet of the reactor is as follows:
MAPD: 1,000 ppm
C6+ (oligomers): 0.43%
Propane: 4.08%
Propylene: 94.79%

The process according to this example therefore makes it possible to hydrogenate the olefinic feedstock with a selectivity of 80.8% of propylene relative to MAPD.

### Example 2

### According to the Invention

The same olefinic feedstock as the one treated in Example 1 (for comparison) has been treated by a hydrogenation process according to the invention, comprising two reactors in series, as illustrated in FIG. 2. The same catalyst mass as the one used in the primary reactor of Example 1 was distributed with half in the first reactor and half in the second reactor, in such a way as to compare the process of Example 1 (for comparison) and that of Example 2 (according to the invention) with the same active catalyst content.

The operating conditions are as follows:
Flow rate of feedstock: 50 m³/h
Composition of the gaseous phase comprising hydrogen: 93% H2, 7% CH4
Total flow rate of hydrogen: 740 Nm³/h of (H2+CH4)
VVH, defined as the ratio of the volumetric flow rate of fresh feedstock at 15° C. to the volume of catalyst: 30 h⁻¹ in each reactor. The increase in the VVH is imposed by the reduction of the volume of catalyst in each reactor.
Diameter of the reactors of 1,080 mm,
Flow rate of recycling: 50.8 m³/h
Absolute pressure in the reactors: 23 bar.
Entrance temperature in the 1^{st} reactor: 30° C.
Entrance temperature in the 2^{nd} reactor: 35° C.

The objective in this example is to lower the content of MAPD (methyl acetylene and propadiene) to 1,000 ppm at the outlet of the 2^{nd} reactor. 50% of the desired overall MAPD conversion (intake concentration brought to 1,000 ppm) is done in each of the reactors.

The composition of the feedstock at the outlet of the 2^{nd} reactor is as follows:
MAPD: 1,000 ppm
C6+ (oligomers): 0.31%
Propane: 4.18%
Propylene: 94.98%.

The process according to the invention (Example 2) therefore makes it possible to hydrogenate the olefinic feedstock with a selectivity of 83.5% of propylene relative to MAPD. A very significant gain is furthermore observed on the final oligomer content, which makes it possible to increase the cycle time in the process according to the invention.

### Example 3

### According to the Invention

A doubled flow rate of feedstock and a composition that is identical to the olefinic feedstock that is treated in Example 1 (for comparison) was treated by a hydrogenation process according to the invention, comprising two reactors in series, as illustrated in FIG. 2. The same overall catalyst mass (active+inactive) as the one used in the reactor of Example 1 was distributed half in the first reactor and half in the second reactor, in such a way as to compare the process of Example 1 (for comparison) and that of Example 2 (according to the invention) with the same catalyst content. This time, the VVH was kept identical to the one used in Example 1 (for comparison).

The operating conditions are as follows:
Feedstock flow rate: 100 m³/h
Composition of the gaseous phase comprising hydrogen: 93% H2, 7% CH4
Total flow rate of hydrogen: 1,480 Nm³/h (H2+CH4)
VVH, defined as the ratio of the volumetric flow rate of fresh feedstock at 15° C. to the volume of catalyst: 15 h⁻¹ in each reactor.
Diameter of the reactors of 1,520 mm,
Flow rate of recycling: 101.6 m³/h
Absolute pressure in the reactors: 23 bar.
Entrance temperature in the 1^{st} reactor: 30° C.
Entrance temperature in the 2^{nd} reactor: 35° C.

The objective in this example is to lower the content of MAPD (methyl acetylene and propadiene) to 1,000 ppm at the outlet of the 2^{nd} reactor. 50% of the desired overall MAPD conversion (intake concentration brought to 1,000 ppm) is done in each of the reactors.

The composition of the feedstock at the outlet of the 2^{nd} reactor is as follows:
MAPD: 1,000 ppm
C6+ (oligomers): 0.31%
Propane: 4.18%
Propylene: 94.98%

The process according to the invention (Example 3) therefore makes it possible to hydrogenate the olefinic feedstock with a selectivity of 83.5% of propylene relative to MAPD.

In addition, by using all of the available catalyst, it is possible to treat a double flow rate of feedstock, without loss of selectivity. The process according to this invention is therefore particularly advantageous when it is desired to increase, at lower cost, the capacity of an existing unit (revamping).

### Example 4

### According to the Invention

The same olefinic feedstock as the one treated in Example 1 (for comparison) has been treated by a hydrogenation process according to the invention, comprising two reactors in series, as illustrated in FIG. 2, each reactor of a size that is two times smaller than the size of the reactor that is used in Example 1 (for comparison). The same catalyst mass as the one used in the reactor of Example 1 has been distributed, half in the first reactor and half in the second reactor, in such a way as to compare the process of Example 1 (for comparison) and the one of Example 2 (according to the invention) with the same catalyst content. This time, the flow rate of H2 was not limited, and the hydrogenation rate was not fixed at 1,000 ppm.

The operating conditions are as follows:
Flow rate of feedstock: 50 m³/h
Composition of the gaseous phase comprising hydrogen: 93% H2, 7% CH4
Total hydrogen flow rate: 751 Nm³/h (H2+CH4)
VVH, defined as the ratio of the volumetric flow rate of feedstock to the volume of catalyst: 30 h⁻¹ in each reactor.
Diameter of the reactors of 1,070 mm,
Flow rate of recycling: 50 m³/h
Absolute pressure in the reactors: 23 bar.
Entrance temperature in the 1^{st} reactor: 30° C.
Entrance temperature in the 2^{nd} reactor: 35° C.

The composition of the feedstock at the outlet of the 2^{nd} reactor is as follows:
MAPD: 570 ppm (50.3% of the desired overall conversion in the first reactor, 49.7% in the second reactor)
C6+ (oligomers): 0.32%
Propane: 4.20%
Propylene: 94.99%.

The process according to the invention (Example 4) therefore makes it possible to hydrogenate the olefinic feedstock with a selectivity of 83.0% of propylene relative to MAPD.

Thus, at the same content of catalyst and hydrogen, for the same flow rate of treated feedstock, the process according to the invention makes it possible to increase the MAPD conversion by maintaining a good selectivity.

From the foregoing description, one skilled in the art can easily ascertain the essential characteristics of this invention and, without departing from the spirit and scope thereof, can make various changes and modifications of the invention to adapt it to various usages and conditions.

1. Process for selective hydrogenation of an unsaturated olefinic feedstock that comprises 3 or 4 carbon atoms or less, in which under hydrogenation conditions, said unsaturated olefinic feedstock and a gaseous phase comprising hydrogen are made to pass over a hydrogenation catalyst, in at least two fixed-bed hydrogenation reactors, each containing at least one catalytic bed, with said hydrogenation reactors being arranged in series to be used in a cyclic manner by repeating, after a stage a) during which the feedstock successively passes through all of the hydrogenation reactors, for a period that is at most equal to the deactivation time of one of said reactors,
successively stages b), b′), and c) defined below:
A stage b), during which the feedstock is introduced into the non-deactivated reactor located immediately downstream, relative to the direction of circulation of the feedstock, from the deactivated reactor, by short-circuiting the deactivated reactor, for a period that is at most equal to the deactivation time of said downstream reactor,
A stage b′), simultaneous to stage b), during which the catalyst of the deactivated reactor is regenerated and/or replaced by fresh catalyst,
A stage c), during which the feedstock successively passes through all of the hydrogenation reactors, with the reactor whose catalyst has been regenerated in stage b′) being reconnected in such a way as to be located downstream from the other reactors relative to the direction of circulation of the feedstock, and said stage being carried out for a period that is at most equal to the deactivation time of a reactor.

2. Process according to claim 1, comprising, before stage b), an additional stage a′) during which a portion of the unsaturated olefinic feedstock supplies an upstream reactor and the remainder of the feedstock supplies at least one of the reactors located downstream from said upstream reactor, for a period that is at most equal to the deactivation time of one of said reactors.

3. Process according to claim 1, in which, during stage b), a quantity of feedstock of 50 to 100% by weight of the quantity of fresh feedstock introduced in stage a), preferably from 60 to 100% by weight, and more preferably from 80 to 100% by volume, is introduced into the non-deactivated reactor.

4. Process according to claim 1, in which the gas/liquid mixture that is obtained from an upstream hydrogenation reactor passes through a heat exchanger (cooler) before being sent to the inlet of a reactor that is located downstream, preferably a reactor that is located immediately downstream.

5. Process according to claim 1, in which the gaseous phase comprising hydrogen is preferably at least introduced at the top of the first reactor through which the feedstock passes and can advantageously also be introduced at the top of each hydrogenation reactor, before or after the exchanger when the latter is present.

6. Process according to claim 1, in which a portion of the gas/liquid effluent that is obtained from a downstream hydrogenation reactor is returned at the inlet of said reactor and/or a reactor that is located upstream, preferably a reactor that is located immediately upstream.

7. Process according to claim 1, in which a portion of the liquid phase that contains the hydrogenated olefinic feedstock that is recovered after the last cycle is recycled in a mixture with the feedstock that is to be hydrogenated.

8. Process according to claim 7, in which the overall recycling rate, defined as the ratio of the volumetric flow rate of recycling to the volumetric flow rate of fresh feedstock entering the reactor, is from approximately 1:10 to approximately 3:1, preferably 1:3 to 2:1.

9. Process according to claim 6, in which the entirety or the non-recycled part of the liquid phase that contains the hydrogenated olefinic feedstock that is recovered after the last cycle is sent to a finishing section, comprising:
A “splitter,” or
A “finishing reactor” preceded by a static mixer M2 for mixing again the hydrogenated olefinic feedstock with a gaseous phase that comprises hydrogen.

10. Process according to claim 1, in which the hydrogenation is done in gas-liquid flow under bubbling conditions with a volumetric vaporization rate at the reactor inlet that ranges from 1 to 50% by volume, preferably ranging from 5 to 30% by volume, or in a liquid-only flow, with a volumetric vaporization rate at the reactor inlet that ranges from 0 to 5% by volume.

11. Process according to claim 1, in which the unsaturated olefinic feedstock comprises olefins having 3 carbon atoms, with the olefinic feedstock preferably comprising 60% to 95% by weight of propylene, and 2 to 8% by weight of polyunsaturated compounds such as methyl acetylene (MA) and propadiene (PD), with the make-up to 100% being essentially propane.

12. Process according to claim 1, in which the unsaturated olefinic feedstock comprises olefins having 4 carbon atoms, with the olefinic feedstock preferably comprising 30 to 50% butadiene.

13. Process according to claim 1, in which at least one of the reactor(s) contains at least two catalyst beds, and in which the gaseous phase that comprises hydrogen is introduced in part in a mixture with the unsaturated olefinic feedstock before the first catalyst bed and in part before the following bed(s) contained in said reactor.

14. Process according to claim 1, in which the proportion of hydrogen in the gaseous feedstock is in particular from 60% to 100% by weight, and most often from 80% to 99.99% by weight, with the make-up to 100% being an inert gas that can be selected from the group that is formed by methane, ethane, propane, butane, nitrogen, argon, carbon monoxide and carbon dioxide (several ppm).

15. Process according to claim 1, in which the flow rate of the gaseous phase comprising hydrogen is adjusted at the inlet of each reactor in such a way as to obtain the desired overall MAPD conversion level.

16. Process according to claim 1, in which the liquid feedstock and the gaseous phase comprising hydrogen introduced at the top of one of the reactors pass through a static mixer before being introduced into said reactor.

17. Process according to claim 1, in which the hydrogenation catalysts that are used are identical or different in each hydrogenation reactor and comprise at least one noble metal of group VIII, preferably palladium, deposited in a crust on a mineral substrate, preferably alumina.

18. Process according to claim 17, in which the catalyst contains at least one doping agent that belongs to the column IB of the periodic table that is selected from the group that is formed by gold, silver, and copper, preferably silver, in a quantity that ranges from 1 to 10,000 ppm by weight, relative to the substrate.

19. Process according to claim 1, in which the hydrogenation is performed, in each reactor, at a temperature that ranges from 10° C. to 80° C., more preferably from 15 to 65° C., and at a pressure from 10 to 40 bar, more preferably from 15 to 35 bar.

20. Process according to claim 1, in which the overall hourly volumetric flow rate (VVH), defined as the ratio of the volumetric flow rate of the fresh feedstock at 15° C. to the total mass of catalyst present in all of the switchable reactors that are used, is generally from 2 h⁻¹ to 100 h⁻¹, preferably from 5 h⁻¹ to 50 h⁻¹, and more preferably from 10 h⁻¹ to 30 h⁻¹.
